(19) United States
(12) Patent Application Publication
(10) Pub. No.: US 2024/0374548 A1
(43) Pub. Date: Nov. 14, 2024

(54) USE OF SNAT2 COMPETITIVE INHIBITOR OR GENE EXPRESSION INHIBITION IN THE PREPARATION OF DRUGS FOR THE PREVENTION AND/OR TREATMENT OF HYPERTENSION

(71) Applicant: EAST CHINA NORMAL UNIVERSITY, Shanghai (CN)

(72) Inventors: Xiaoyan ZHANG, Shanghai (CN); Youfei GUAN, Shanghai (CN); Chunxiu DU, Shanghai (CN); Hu XU, Shanghai (CN)

(21) Appl. No.: 18/747,728

(22) Filed: Jun. 19, 2024

Related U.S. Application Data
(63) Continuation of application No. PCT/CN2023/111949, filed on Aug. 9, 2023.

(30) Foreign Application Priority Data
Sep. 22, 2022 (CN) 202211156273.6

(51) Int. Cl. A61K 31/198; A61K 38/46; A61K 48/00; A61P 9/12; C12N 5/071; G01N 33/50

(52) U.S. Cl. CPC A61K 31/198; A61K 38/465; A61K 48/005; A61P 9/12; C12N 5/069; G01N 33/5064; C12N 2510/00

(57) ABSTRACT

The present invention discloses the use of SNAT2 competitive inhibitors or gene expression inhibition in the preparation of drugs for the prevention and/or treatment of hypertensive disorders. The SNAT2 competitive inhibitor is α-aminoisobutyric acid (MeAIB). The present invention demonstrates that MeAIB, a competitive inhibitor of SNAT2, and knockout of the SNAT2 gene can prevent and treat hypertension, and is of significant value in the prevention and treatment of primary hypertension. The present invention provides evidence showing that SNAT2 serves as a potential target for drug discovery and an intervention tool in primary hypertension.

### RELATED APPLICATIONS

The present application is a Continuation of International Application Number PCT/CN2023/111949 filed Aug. 9, 2023, which claims priority to Chinese Application Number 202211156273.6 filed Sep. 22, 2022, the disclosures of which are hereby incorporated by reference herein in their entireties.

### TECHNICAL FIELD

The present invention relates to the technical field of biomedicine, and particularly to the application of SNAT2 (SLC38a2) competitive inhibitor or gene expression inhibition in the preparation of drugs for the prevention and/or treatment of primary hypertension and its relevant disorders.

### BACKGROUND

Primary hypertension is a cardiovascular syndrome characterized by elevated systemic arterial blood pressure in the physical circulation, often referred to simply as hypertension. Hypertension is a growing public health problem worldwide, and is usually defined as a cardiovascular disease in which diastolic blood pressure is higher than 90 mmHg and systolic blood pressure is higher than 140 mmHg. By 2015, 1.15 billion people worldwide is suffering from hypertension. Hypertension poses serious dangers and can lead to complications such as cerebral hemorrhage, cerebral infarction, fundus blindness, myocardial infarction and kidney disease.

The heart, kidneys and blood vessels are the main target organs for the psychophysiology effects of hypertension, and there may be no obvious pathological changes in the early stage. The cardiac changes caused by long-term hypertension are mainly left ventricular hypertrophy and enlargement; and the systemic small arterial lesions are mainly the increase in the wall-to-lumen ratio and the narrowing of lumen diameter, leading to ischemia of the important target organs such as the heart, the brain, the kidneys and other tissues. Prolonged hypertension and accompanying risk factors may promote the formation and development of atherosclerosis, ultimately leading to coronary heart disease (angina pectoris, myocardial infarction) and stroke associated with coronary or cerebrovascular function and structural remodeling, posing a serious threat to one's health. It is currently believed that vascular endothelial dysfunction is the earliest and most important vascular injury caused by hypertension.

Clinical evidence suggests that a decrease in systolic blood pressure of 10-20 mmHg or a decrease in diastolic blood pressure of 5-6 mmHg is associated with a reduction in stroke, coronary heart disease, and cardiovascular mortality of 38%, 16%, and 20%, respectively, and a reduction in heart failure of more than 50% over a 3-5-year period. The ultimate goal of antihypertensive treatment is to reduce the incidence of cardiac, cerebral, and vascular diseases and mortality and renal complications in hypertensive patients. Current antihypertensive drugs can be categorized into five main groups, namely diuretics, β receptor antagonists, calcium channel blockers (CCB), angiotensin-converting enzyme inhibitors (ACEI) and angiotensin II receptor antagonists (ARB) and the likes. However, these drugs are mainly used to alleviate symptoms, and less helpful to the overall prognosis of the disease, and are less effective in significantly improving pathological changes such as vascular remodeling; moreover, these drug treatments produce adverse effects such as fatigue, increased urination, heart rate abnormalities, acratia, chills in the extremities, facial flushing, dry cough, and angioedema; therefore, the efficacy and safety of the existing drugs are not satisfactory.

Antimicrobial peptides (AMPs) are the earliest immune active molecules produced by organisms that evolved to adapt to the environment and survive. They are an important part of the natural immune system and play an extremely important role in the host's immune defense against pathogens; therefore, they are known as “natural antibacterial agents”. Unmodified, these peptides are potent, broad-spectrum antibiotics which demonstrate potential as novel therapeutic agents. AMPs have been demonstrated to kill Gram-negative and Gram-positive bacteria (including strains that are resistant to conventional antibiotics), mycobacteria (including Mycobacterium tuberculosis), enveloped viruses, and fungi.

Naturally occurring AMPs are typically short peptides, generally between 12 and 50 amino acids. These peptides often include two or more positively charged residues provided by arginine, lysine or histidine (in acidic environments) and frequently a large proportion (generally >50%) of hydrophobic residues (see, e.g., Papagianni et al. (2003) Biotechnol Adv 21:465; Sitaram and Nagaraj (2002) Curr Pharm Des 8:727; Durr et al. (2006) Biochim. Biophys. Acta 1758:1408-1425).

AMPs have shown unprecedented advantages as promising biomaterials against multidrug-resistant bacteria. Compared to traditional antibiotics, the amphiphilic structure of AMPs can selectively bind to and penetrate cell membranes, thereby destroying the integrity of the cell membrane and killing the pathogen. Without wishing to be bound by any particular theory, it is believed that the bacterial cell membrane is the target of AMPs. Therefore, precisely because of the low likelihood of variation in the cell membrane structure, the probability of sensitive bacteria developing resistance is minimal.

Currently, AMPs are considered ideal candidates to replace antibiotics due to their strong antibacterial potential and unique mechanism of action. So far, more than 3000 AMPs have been recorded in the Antimicrobial Peptide Database (APD), covering functions such as antibacterial, antifungal, antiviral, and antiparasitic.

Despite the advantages, organism-derived AMPs are organism-specific, so it is difficult to directly apply them to the human body as such AMPs might be highly toxic. For this reason, many AMPs cannot be used as drugs. In addition, the structure of linear AMPs is unstable and has proteolytic instability, making it difficult for clinical transformation and application.

### SUMMARY

It is an object of the present invention to provide the use of SNAT2 competitive inhibitors or gene expression inhibition in the preparation of drugs for the prevention and/or treatment of primary hypertension and its relevant disorders (angina pectoris, myocardial infarction, stroke, etc.).

Further, it is an object of the present invention to provide the use of a substance having SNAT2 competitive inhibitory activity or a means for inhibiting its expression at the level of gene transcription in the preparation of drugs for the prevention and/or treatment of primary hypertension and its relevant disorders (angina pectoris, myocardial infarction, stroke, etc.).

Further, it is an object of the present invention to provide the use of a substance having SNAT2 gene and its products (mRNAs and proteins) inhibitory activity or capable of knocking out or silencing the SNAT2 gene in the preparation of drugs for the prevention and/or treatment of primary hypertension and its relevant disorders (angina pectoris, myocardial infarction, stroke, etc.).

The substance having SNAT2 competitive inhibitory activity may specifically be α-aminoisobutyric acid (MeAIB).

More preferably, the drug is a drug having any one of the following functions:
1) a drug for lowering a level of blood pressure in a basal state;
2) a drug for the prevention and/or treatment of hypertension; and
3) a drug promoting a production of a vasodilator NO.

Preferably, the drug is made with a SNAT2 competitive inhibitor or a substance with SNAT2 competitive inhibitory activity as an active ingredient, and may comprise pharmaceutically acceptable excipients.

Preferably, the drug is a systemic or localized therapeutic drug and means that targets the SNAT2 gene and its products (mRNAs and proteins) for intervention.

Preferably, the drug may comprise dosage forms such as powder, paste, granule, pill, tablet, capsule, granule, oral thick paste, decoction, spray administration or injection administration.

On the basis of conforming to the common sense in the field, each of the above preferred conditions may be arbitrarily combined without exceeding the conception and protection scope of the present invention.

The present invention also provides a method of preparing a cell model for screening a drug for lowering blood pressure, comprising the steps of:
1) obtaining vascular endothelial cells from an animal;
2) treating the vascular endothelial cells with the substance with SNAT2 gene and its products (mRNAs and proteins) inhibitory activity or capable of knocking out or silencing the SNAT2 gene, thereby obtaining vascular endothelial cells in which the expression of the SNAT2 gene and its products (mRNAs and proteins) is reduced, or the SNAT2 gene is knocked out or silenced; and
3) determining a content of NO in the vascular endothelial cells obtained in the step 2) with a reduced expression of the SNAT2 gene and its products (mRNAs and proteins), or with a knocked-out or silenced SNAT2 gene, as an indicator reflecting the level of blood pressure.

The animal may specifically be a wild type mouse, and more specifically, may be a wild type C57BL/6 mouse;

The substance with SNAT2 gene and its products (mRNAs and proteins) competitive inhibitory activity may be α-aminoisobutyric acid (MeAIB);

The content of NO in vascular endothelial cells was determined using a total nitric oxide assay kit.

The cell model for screening blood pressure-lowering drugs obtained by the preparation method described above.

A method for screening a blood pressure-lowering drug using the above cell model, comprising the following steps:
1) setting up groups: test drug group, positive control group and blank control group are set up, respectively, wherein the positive control group is treated by administrating arginine, and the blank control group is treated by administrating an equal volume of PBS;
2) treating the cell models in each group using each group of drugs; and
3) determining the content of NO in the treated cells, if the content of NO obtained in the group treated by the test drug is higher than or equal to that obtained in the positive control group, and there is a statistically significant difference with respect to the blank control group, the test drug has blood pressure lowering activity; and if the content of NO obtained in the group treated by the test drug is lower than that obtained in the positive control group, and there is no statistically significant difference from the blank control group, the test drug has no blood pressure lowering activity.

The present invention has verified that SNAT2 inhibition and knockout or silence can improve the physical manifestations of primary hypertension for the first time, in specifically terms of lowering the blood pressure in the basal state (basal vascular resistance), resisting the elevation of blood pressure caused by a high salt diet (salt sensitivity), and lowering the blood pressure level in animals with hypertension. Therefore, SNAT2 genes and proteins can be utilized as potential targets for drug development in primary hypertension, and relevant research animal and cell models can be prepared for screening antihypertensive drugs.

It is found in the present invention that MeAIB, as a competitive inhibitor of SNAT2, has an important value for application in the prevention and treatment of primary hypertension.

In addition, it is found in the study that SNAT2 is very highly expressed at the vascular endothelium, and MeAIB, a competitive inhibitor of SNAT2, may significantly reduced blood pressure in wild-type mice, and the blood pressure of the mice having SNAT2 systemic knocked out and vascular endothelium-specific knocked out is significantly lower than that of the wild-type mice. This finding provides a theoretical and experimental basis for the screening of drugs for the prevention and/or treatment of primary hypertensive disorders by specifically inhibiting SNAT2 expression at the vascular endothelium.

### BRIEF DESCRIPTION OF THE DRAWINGS

FIG. 1 is a schematic diagram showing the result of SNAT2 competitive inhibitor (MeAIB) in Example 1 of the present invention that significantly reduced basal blood pressure levels (SBP, systolic blood pressure) in wild-type mice (7 wild-type mice in total, MeAIB was dissolved in water to a final concentration of 1 g/L, and blood pressure was reduced in the mice after 2 weeks of water consumption of MeAIB, and the results are expressed as the mean±standard error of the mean, and * indicates p<0.05).

FIGS. 2A and 2B are schematic diagrams of SNAT2 systemic knockout mice (SNAT2−/−) obtained by CRISPR/Cas9 by deletion of 10 bp (GCGATTGTGG) at Exon4 resulting in a frameshift mutation in Example 2 of the present invention (FIG. 2A), and DNA sequencing results (FIG. 2B).

FIGS. 3A-3C are schematic diagrams showing the results of systemic knockout of SNAT2 (SNAT2−/−) in Example 2 of the present invention significantly reducing basal blood pressure levels in mice (in which FIG. 3A shows the systolic blood pressure (SBP); FIG. 3B shows the diastolic blood pressure; and FIG. 3C shows the mean arterial pressure (MAP). There are approx. 30 mice in each group, the results are expressed as the mean±standard error of the mean, and *** indicates p<0.001).

FIGS. 4A and 4B are schematic diagrams of the flox modification of SNAT2 (Slc38a2) gene at both ends of exons 5 and 10 using homologous recombination of the fertilized egg by the principle of homologous recombination in Example 3 (FIG. 4A) and genetically identified by DNA gel electrophoresis (FIG. 4B).

FIGS. 5A-5C are schematic diagrams showing the results of endothelial-specific knockout of SNAT2 (EC-SNAT2-cKO) in Example 3 of the present invention significantly reducing basal blood pressure levels in mice (in which FIG. 5A shows the systolic blood pressure; FIG. 5B shows the diastolic blood pressure; and FIG. 5C shows the mean arterial pressure. (There are approx. 13 mice in each group, the results are expressed as the mean±standard error of the mean, and ** indicates p<0.01, *** indicates p<0.001).

FIG. 6 is a schematic diagram showing the result of knockout of SNAT2 in Example 4 of the present invention significantly ameliorating the increase in blood pressure due to high salt diet (there are 4-8 mice in each group, results are expressed as the mean±standard error of the mean, and * indicates p<0.05, ** indicates p<0.01).

FIG. 7 is a schematic diagram showing the results that knockout (KO) of SNAT2 in Example 5 of the present invention significantly increased serum levels of the vasodilator NO in mice (6 mice per group, serum NO levels were measured. Results are expressed as mean±standard error, * indicates p<0.05).

FIGS. 8A-8C shows that MeAIB, an inhibitor of SNAT2, dose-dependently increased NO production and endothelial NO synthase activity in human umbilical vein endothelial cells (HUVEC) in Example 6 of the present invention (among them, FIG. 8A: optical microscopy shows changes in cell morphology after MeAIB dose-dependent treatment of the HUVEC cells; FIG. 8B: MeAIB dose-dependent treatment; and FIG. 8B: measurement of supernatant NO content after MeAIB dose-dependent treatment. FIG. 8B: MeAIB dose-dependent treatment of HUVEC cells; FIG. 8B: measurement of NO content in the supernatant of the HUVEC cells after MeAIB dose-dependent treatment; FIG. 8C: detection of endothelial NO synthase (eNOS) and its phosphorylated protein (p-eNOSSer1177) expression after MeAIB dose-dependent treatment of HUVEC cells detected by western blot. (The experiment was repeated 3 times, and the results are expressed as mean±standard error of the mean, ** indicates p<0.01, *** indicates p<0.001).

### DETAILED DESCRIPTION

The The methods used in the following examples are conventional methods unless otherwise stated.

The present invention is further described below in conjunction with the examples without limiting the invention in any way, and any changes or improvements made based on the teachings of the present invention are within the scope of protection of the present invention.

The specific technical solutions adopted are as follows:
High salt diet-induced hypertension model: mice were fed a high salt diet (Medicience Ltd) containing 3.5% (this is a concentration by mass, 100 g of grain contains 3.5 g NaCl) NaCl for 28 days to induce a mouse hypertension model.
Measurement of blood pressure by tail-cut method in mice: A noninvasive tail sleeve instrument (BP-2010 series blood pressure meter, Softron) was used for blood pressure measurement in mice. The sensor was placed around the tail of the mouse, and the blood flow signal was monitored by inflating and deflating the tail artery while pressurizing and releasing the pressure to obtain the blood pressure value. The method is noninvasive and does not require surgery. The animals were pre-trained for 2 weeks to fully acclimatize to the environment. Before recording, the mouse is rested for no less than 10 minutes until it is comfortable and quiet in its cage.
Human Umbilical Vein Endothelial Cells (HUVECs) Culture: Umbilical cords within 24 hours of delivery were placed in sterile 1× Hepes buffer; blood and buffer were gently wiped off the umbilical cords with sterilized gauze; the end of the umbilical cords were dried and the umbilical veins were sought out; a metal needle was inserted into the umbilical vein and clamped with hemostatic forceps, and the metal needle was mounted on a 50 ml syringe filled with 1× Hepes buffer, then the umbilical vein was rinsed with Hepes buffer repeatedly to ensure that it is clean; after it is clean, the metal needle was inserted into the other end of the umbilical vein and secured with hemostatic forceps; trypsin was slowly injected into the umbilical cord, and when the trypsin reaches the hemostatic forceps, seal it with a 1 ml syringe, and the rest of the trypsin was injected into the umbilical cord; the umbilical cord was placed into a sterilized cup containing approximately 20 mL of prewarmed 1× Hepes buffer, the umbilical cord was then incubated in a 37° C. water bath cauldron for 10 min, followed by placed in a 50 mL centrifuge tube containing 5 mL of endothelial cell (EC)-medium; the hemostat was carefully released and the umbilical cord was rinsed with 20 mL of buffer-containing syringe; the cell suspension was inoculated in a T25 culture flask pre-coated with collagen from rat tail, and incubated in an incubator at 37° C. containing 5% CO2 (change the medium after 2 h); after about 3-6 days of full growth, the cells were transferred to T75 culture flasks for secondary culture, which was the first generation (P1), and the cells of P3-P9 generations were used for cell experiments.
Determination of the content of Nitric Oxide (NO): nitrate reductase was used in the Total Nitric Oxide Detection Kit to reduce nitrate to nitrite, and then nitrite was measured by the classic Griess reagent, so as to determine the content of total Nitric Oxide. Nitric Oxide itself is extremely unstable, and will be metabolized to nitrate and nitrite very quickly in the cell. The total amount of nitrate and nitrite measured by the above method can be used to calculate the total amount of nitric oxide.
Molecular biology experiments: Detection and analysis were carried out by Western Blot and other techniques.

### EXAMPLE 1

In this Example, SNAT2 competitive inhibitor (MeAIB) was found to be capable of reducing blood pressure levels in the basal state of wild-type mice.

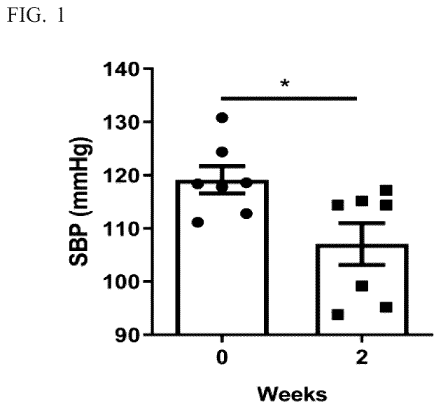

Primary hypertension is a cardiovascular syndrome characterized by elevated arterial blood pressure in the physical circulation, often referred to simply as hypertension. Hypertension is usually defined as a cardiovascular disease in which systolic blood pressure is higher than 140 mmHg and/or diastolic blood pressure is higher than 90 mmHg. In this study, wild-type C57BL/6 mice (Liaoning Changsheng biotechnology co., Ltd.) were selected for the detection of basal blood pressure by the tail-cut method, and then MeAIB (1 g/L) was given to the mice in the drinking water for 2 weeks, and then the blood pressure (systolic blood pressure, SBP) was measured after drinking water. The results showed that the systolic blood pressure of mice decreased after drinking water containing MeAIB (FIG. 1).

### EXAMPLE 2

In this Example, knockout of the SNAT2 gene (SNAT2−/−) leading to the decrease of blood pressure (systolic blood pressure, diastolic blood pressure and mean arterial pressure) in mice at the basic state was studied.

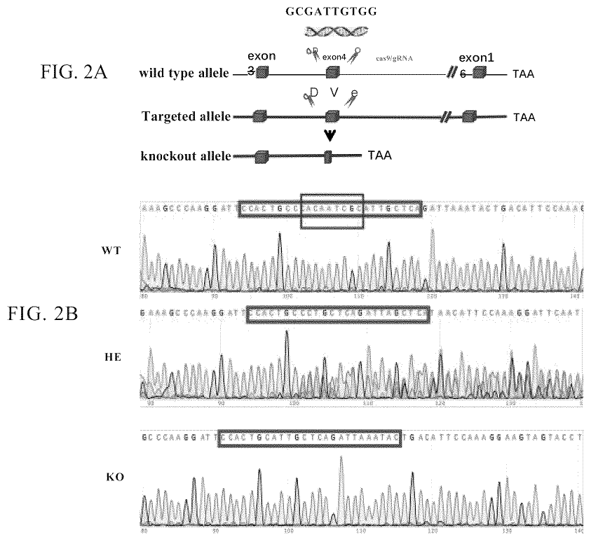

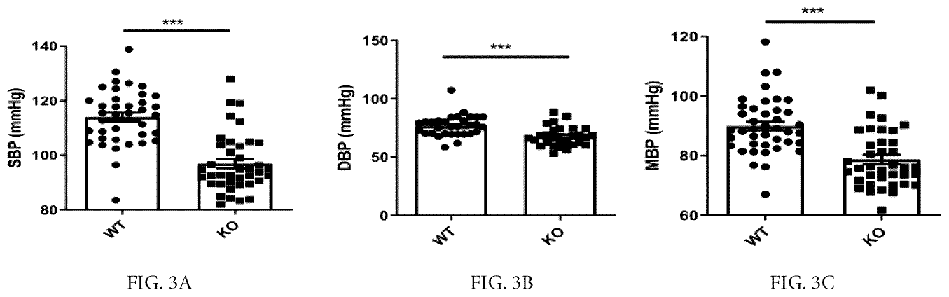

SNAT2 systemic knockout mice were obtained by CRISPR/Cas9 by deletion of 10 bp (GCGATTGTGG) at Exon4 resulting in a frameshift mutation (see FIG. 2A), and identified by DNA sequencing (see FIG. 2B). SNAT2 knockout mice under the C57BL/6 background have homozygous lethal phenotype. In order to obtain sufficient numbers of SNAT2 WT and systemic SNAT2 knockout (KO) mice, SNAT2 heterozygous males from the C57BL/6 background backcrossed with females from the wild-type 129 background, and the adult mice obtained after 5 generations of backcrossing were used for subsequent experiments. Compared with wild-type mice (SNAT2+/+), SNAT2 knockout mice (SNAT2−/−) had lower systolic blood pressure (SBP, FIG. 3A), diastolic blood pressure (DBP, FIG. 3B), and mean arterial pressure (MBP, FIG. 3C).

### EXAMPLE 3

In this Example, endothelial-specific knockout of SNAT2 (EC-SNAT2-cKO) leading to the reduction of blood pressure (systolic, diastolic, and mean arterial pressure) in the basal state of mice was studied.

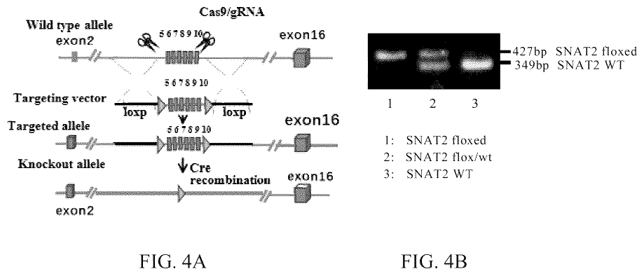

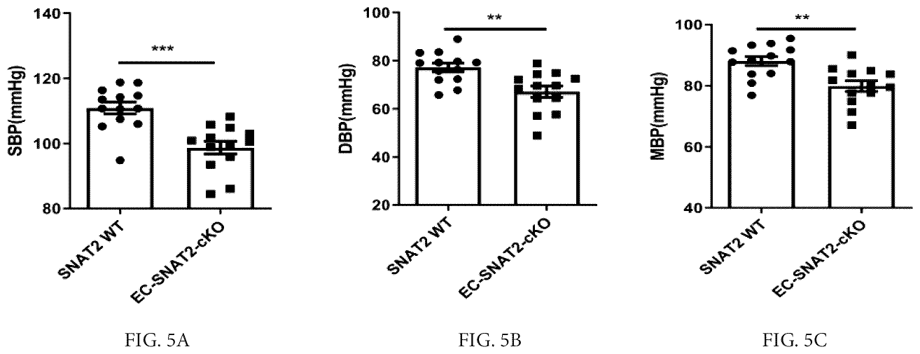

Using the principle of homologous recombination, flox modification was carried out on the SNAT2 (Slc38a2) gene at both ends of exons 5 and 10 thereof by homologous recombination of fertilized eggs and genetically identified by DNA gel electrophoresis (see FIG. 4). To further elucidate the role of SNAT2 at vascular endothelial in blood pressure regulation, we crossed SNAT2 flox/flox mice with vascular endothelial-specific Cre mice (VE-Cadherin-Cre) (The Jackson Laboratory, 017968) to obtain endothelial-specific SNAT2 knockout mice (EC-SNAT2-cKO). We then measured the blood pressure of the mice using the tail-cut method. The systolic blood pressure (SBP, FIG. 5A), diastolic blood pressure (DBP, FIG. 5B), and mean arterial pressure (MBP, FIG. 5C) of endothelial-specific SNAT2 gene knockout mice (EC-SNAT2-cKO) were significantly lower than those of wild-type mice (SNAT2+/+, WT).

### EXAMPLE 4

In this Example, the resistance of SNAT2 gene knockout to the increase of blood pressure (systolic blood pressure) in mice caused by high salt diet was studied.

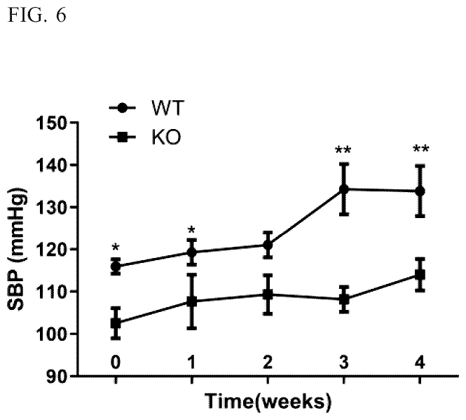

In this study, mice were divided into wild-type (SNAT2+/+) and SNAT2 knockout mice (SNAT2−/−), and blood pressure was measured in the basal state of the mice by the tail-cut method, and then the mice were given a high-salt (3.5% NaCl) diet for 4 weeks, and blood pressure was measured weekly. It was found that systolic blood pressure (SBP) was increased in SNAT2+/+mice after the high-salt diet, whereas SNAT2−/− mice were resistant to the high-salt diet-induced increase in systolic blood pressure in mice (FIG. 6).

### EXAMPLE 5

In this Example, the increase in serum NO in mice resulting from knockout of the SNAT2 gene was studied.

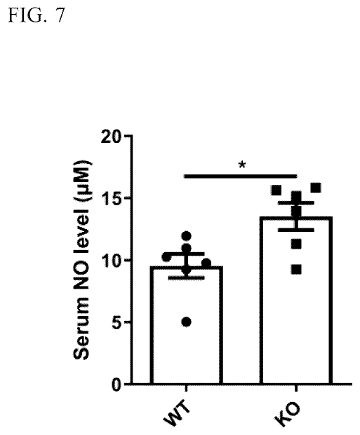

In this study, mice were divided into wild-type (SNAT2+/+) and SNAT2 knockout mice (SNAT2−/−). Blood was collected from the angular vein of the mice, and then centrifuged at 3,000 rpm for 10 min after standing at room temperature for 2 h. The supernatant was taken as serum. The total NO content of serum was measured by Griess reagent, and it was found that the serum NO content was increased in SNAT2−/− mice (FIG. 7).

### EXAMPLE 6

In this Example, it was studied that MeAIB, a competitive inhibitor of SNAT2, can dose-dependently increase NO levels in human umbilical vein endothelial cells (HUVEC).

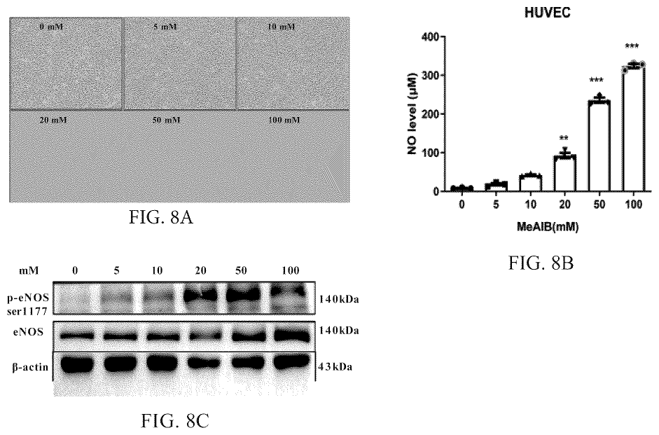

Human umbilical vein endothelial cells were cultured (FIG. 8A), and when the cell fusion reached 80%, it was given MeAIB dose-dependent treatment (0, 5, 10, 20, 50, and 100 mM) for 24 h. The NO content in cell supernatant was measured in the supernatant of the cells by Griess reagent, and it was found that MeAIB increased the NO content in the supernatant of the cells dose dependently (FIG. 8B), and MeAIB increased the eNOS and p-eNOS of the cells by Western Blot (FIG. 8B). Western Blot was used to detect the protein expression of eNOS and p-eNOS (Ser1177) in the cells, and the results showed that the protein expression of p-eNOS (Ser1177) was increased (FIG. 8C), indicating that the activity of eNOS, which is related to the synthesis of NO, was increased.

### INDUSTRIAL APPLICATIONS

It is figured out in the present invention that MeAIB, as a competitive inhibitor of SNAT2, has an important value for application in the prevention and treatment of primary hypertension. SNAT2 is found to be very highly expressed at the vascular endothelium, and MeAIB, a competitive inhibitor of SNAT2, may significantly reduce blood pressure in wild-type mice, and the blood pressure of the mice having SNAT2 systemic knocked out and vascular endothelium-specific knocked out is significantly lower than that of the wild-type mice. This finding provides a theoretical and experimental basis for screening drugs for the prevention and/or treatment of primary hypertensive disorders by specifically inhibiting SNAT2 expression at the vascular endothelium.

1-10. (canceled)

11. A method for preventing and/or treating primary hypertension and its relevant disorders using an SNAT2 competitive inhibitor or using a substance with SNAT2 competitive inhibitory activity or using a substance with SNAT2 gene and its products (mRNAs and proteins) inhibitory activity or capable of knocking out or silencing the SNAT2 gene.

12. The method according to claim 11, wherein the substance with SNAT2 competitive inhibitory activity is α-aminoisobutyric acid (MeAIB).

13. The method according to claim 11, wherein the relevant disorder of the primary hypertension comprises angina pectoris, myocardial infarction and stroke.

14. The method according to claim 11, wherein the substance with SNAT2 gene and its products (mRNAs and proteins) inhibitory activity or capable of knocking out or silencing the SNAT2 gene is drug of small nucleic acid comprising antisense oligonucleotides, small interfering RNAs, micro RNAs and nucleic acid aptamers.

15. The method according to claim 11, wherein the method can:
1) lowering a level of blood pressure in a basal state;
2) preventing and/or treating hypertension; and
3) promoting a production of a vasodilator NO.

16. A method of preparing genetically modified vascular endothelial cells for screening a drug for lowering blood pressure, comprising the steps of:
1) obtaining vascular endothelial cells from an animal or human umbilical cord;
2) treating the vascular endothelial cells with the substance with SNAT2 gene and its products (mRNAs and proteins) inhibitory activity or capable of knocking out or silencing the SNAT2 gene, thereby obtaining genetically modified vascular endothelial cells in which the expression of the SNAT2 gene and its products (mRNAs and proteins) is reduced, or the SNAT2 gene is knocked out or silenced; and
3) determining a content of NO in the genetically modified vascular endothelial cells obtained in the step 2) with a reduced expression of the SNAT2 gene and its products (mRNAs and proteins), or with a knocked-out or silenced SNAT2 gene, as an indicator reflecting the level of blood pressure.

17. Genetically modified vascular endothelial cells obtained by the method of preparation of claim 16.

18. The method for screening a blood pressure lowering drug using the genetically modified vascular endothelial cells of claim 17, comprising the steps of:
1) setting up groups: test drug group, positive control group and blank control group are set up, respectively, wherein the positive control group is treated by administrating an equal volume of arginine, and the blank control group is treated by administrating an equal volume of PBS;
2) treating the cell models in each group using each group of drugs; and
3) determining the content of NO in the treated cells, if the content of NO obtained in the group treated by the test drug is higher than or equal to that obtained in the positive control group, and there is a statistically significant difference with respect to the blank control group, the test drug has blood pressure lowering activity; and if the content of NO obtained in the group treated by the test drug is lower than that obtained in the positive control group, and there is no statistically significant difference from the blank control group, the test drug has no blood pressure lowering activity.
